(19) United States
(12) Patent Application Publication
(10) Pub. No.: US 2021/0012492 A1
(43) Pub. Date: Jan. 14, 2021

(54) SYSTEMS AND METHODS FOR OBTAINING 3-D IMAGES FROM X-RAY INFORMATION FOR DEFORMED ELONGATE BONES

(71) Applicant: Vikas KARADE, Mumbai (IN)

(72) Inventors: Vikas KARADE, Mumbai (IN); Amit MAURYA, Thane (IN)

(21) Appl. No.: 16/981,365

(22) PCT Filed: Mar. 21, 2019
(86) PCT No.: PCT/IN2019/050231

§ 371 Date: Sep. 16, 2020

(30) Foreign Application Priority Data
Mar. 21, 2018 (IN) 201821010314

(51) Int. Cl. G06T 7/00; G06T 7/149; G06T 7/30; G06T 3/40; G06T 11/00; G16H 30/20

(52) U.S. Cl. CPC G06T 7/0012; G06T 7/149; G06T 7/30; G06T 3/40; G06T 2207/10081; G06T 11/006; G16H 30/20; G06T 2207/30008; G06T 2207/20021; G06T 11/005

(57) ABSTRACT

A method for obtaining 3-dimensional images from x-ray information for deformed bones, said method comprising the steps of: acquiring an x-ray image of a bone; determining camera model to determine spatial values of source and a spatial value of bone; acquiring contour points and landmark points, in a 2-dimensional co-ordinate system; transforming said contour points and said landmark points from said 2-dimensional co-ordinate system into a 3-dimensional co-ordinate system of an imaging space; importing a pre-created 3-dimensional template along with anatomical values, anatomical regions, anatomical landmarks, and anatomical axes; aligning 3-dimensional template using identified anatomical landmarks and segmented anatomical regions with respect to camera model in order to obtain an aligned template; and deforming said aligned template to reconstruct deformity in said bone of interest, in such a way that its projection on an X-ray image plane in said imaging space matches exactly with said acquired contour points.

### FIELD OF THE INVENTION:

This invention relates to the field of biomedical engineering.

Particularly, this invention to systems and methods for obtaining 3-D images from X-ray information for deformed elongate bones.

### BACKGROUND OF THE INVENTION:

Surgical planning is a preoperative method of visualising a surgical intervention, to set out the surgical steps and bone segment navigation in the context of computer assisted surgery. Surgical planning is important in orthopedic surgery, neurosurgery, oral and maxillofacial surgery, etc. Execution, or transfer of the surgical planning to the patient, is generally performed with a medical navigation system.

Some orthopedic surgeries, like knee or hip replacement and complex deformity corrections, include cutting or drilling on an irregular-shaped a bone. Performance and accuracy of such surgeries improves if the surgery is planned pre-operatively. Surgeons are trained to use conventional 2D image data to prepare for their complex procedures. Such planning may be made from X-ray images of CT data sets or the like. CT data sets are large compared to X-ray images. Hard copies of X-ray images of the particular region of the patient's body for operation, such as a knee or hip-joint, or digital X-ray images on a PC based, can be used for 2D operational planning.

### SUMMARY OF THE INVENTION:

Example embodiments include computer systems for transforming 2D anatomical X-ray images into 3D renderings for surgical preparation through example methods. Such methods include taking x-ray image of body part to be converted to 3D and determining a camera model of the x-ray image. For example, spatial values of the X-ray source and body part may indicate the camera model. A contour of the body part is extracted from the X-ray and analyzed based on its anatomical regions. Each region is assigned 2D anatomical values in the contour. A separate 3D template for the body part is then modified to match the 2D X-ray images by extracting silhouette vertices from the 3D template and their projections, according to the camera model and how those features are initially aligned in the template. The template can then be aligned with the x-ray image and projected on an image plane for the appropriate camera model to obtain a 2D projection model. The template is then modified to match the 2D anatomical values by comparing the 2D projection with the corresponding identified anatomical values. A best matching point on the contour, for each extracted silhouette vertex projection, is identified between the 2D projection and contour. The resulting matching points are then back projected based on camera model to form a back projected ray with target positions that are closest to a corresponding silhouette vertex. The 3D template can then be deformed so that its silhouette vertices match the target positions, resulting in a 3D image that corresponds to the 2D X-ray image.

A limitation of the prior art is that input images required to be orthogonally oriented. Therefore, there is a need for a system and method which can provide a deformed 3D reconstructed image which is a pre-cursory input towards a final 3D reconstructed image.

According to the prior art, the following steps refer to a deformation methodology:

determining a best matching point on said extracted contour, for each of said extracted silhouette vertex projection, for 2-dimensional to 2-dimensional correspondence, of silhouette vertex projection and said extracted contour;

back-projecting each of said best matching points according to said camera model to form a back projected ray, said ray being formed by said source and said best matching point;

determining a target position, said target position being a position, on each of said back projected rays, that is closest to a corresponding silhouette vertex; and

deforming said pre-created 3-dimensional template such that said extracted silhouette vertices achieve positions of their corresponding target positions in order to obtain a 3- dimensional reconstructed image.

Accordingly, an object of the invention is to provide an interim modified 3D reconstructed image which can be further deformed to obtain a final 3D reconstructed image.

Accordingly, another object of the invention is to provide a system and method for modifying an aligned template to match identified 2D values using at least an anatomical value from said image to 2D projection of corresponding identified values of a pre-created 3D template.

### BRIEF DESCRIPTIONS OF THE DRAWINGS:

Example embodiments will become more apparent by describing, in detail, the attached drawings, wherein like elements are represented by like reference numerals, which are given by way of illustration only and thus do not limit the example embodiments herein.

FIG. 1 is an illustration of a schematic block diagram of an example embodiment system.

FIG. 2 is an illustration of a camera model source positioning.

FIG. 3A is an illustration of anatomical regions for femur and tibia.

FIG. 3B is an illustration of anatomical landmarks and the anatomical parameters for femur and tibia.

FIG. 3C is an illustration of anatomical regions corresponding to the regions distinguished in the contour of the X-ray image.

FIG. 3D is an illustration of anatomical landmarks identified based on anatomical regions.

FIG. 4 is an illustration of triangulation of projected points, meshing after putting constraints and the outer contour calculation.

FIG. 5 is an illustration of femur and tibia images wherein with corresponding transformations to the template.

FIG. 6 is an illustration of the template model before and after the alignment.

FIG. 7 is an illustration of template deformation.

FIG. 8 is an illustration of deformation for local matching.

FIG. 9 is an illustration of extraction of separate boundary contours for bone shaft, from an ML view x-ray image.

FIG. 10 is an illustration of template alignment with respect to Medial-Lateral image.

FIG. 11 is a flowchart of an example method of 3D image reconstruction from a single X-ray image.

FIG. 12A is a flowchart of an example method of 3D image reconstruction and template deformation separately with respect to ML and then AP x-ray image.

FIG. 12B is a flowchart of an example method of the 3D image reconstruction and template deformation simultaneously with respect to ML and then AP x-ray image.

FIG. 13 is a flowchart of an example method of determining alignment of the template with respect to the input x-ray image.

FIG. 14 is a flowchart of an example method of 3D image reconstruction from a two Orthogonal X-ray image.

FIG. 15 illustrates the imaging space with calibration parameters.

FIG. 16 depicts landmarks and axes on the bone.

FIG. 17 illustrates computation of landmark based on the standard directions of the bone's anatomical co-ordinate system.

FIG. 18 illustrates computed anatomical landmarks.

FIG. 19 illustrates the 3D reconstruction flowchart.

FIG. 20 illustrates a flowchart for initial template alignment and condyle deformation.

FIG. 21 illustrates a flowchart for deforming a template bone.

FIG. 22 illustrates a flowchart for local deformation.

### DETAILED DESCRIPTION:

Because this is a patent document, general broad rules of construction should be applied when reading it. Everything described and shown in this document is an example of subject matter falling within the scope of the claims, appended below. Any specific structural and functional details disclosed herein are merely for purposes of describing how to make and use examples. Several different embodiments and methods not specifically disclosed herein may fall within the claim scope; as such, the claims may be embodied in many alternate forms and should not be construed as limited to only examples set forth herein.

It will be understood that, although the terms first, second, etc. may be used herein to describe various elements, these elements should not be limited to any order by these terms. These terms are used only to distinguish one element from another; where there are “second” or higher ordinals, there merely must be that many number of elements, without necessarily any difference or other relationship. For example, a first element could be termed a second element, and, similarly, a second element could be termed a first element, without departing from the scope of example embodiments or methods. As used herein, the term “and/or” includes all combinations of one or more of the associated listed items. The use of “etc.” is defined as “et cetera” and indicates the inclusion of all other elements belonging to the same group of the preceding items, in any “and/or” combination(s).

It will be understood that when an element is referred to as being “connected,” “coupled,” “mated,” “attached,” “fixed,” etc. to another element, it can be directly connected to the other element, or intervening elements may be present. In contrast, when an element is referred to as being “directly connected,” “directly coupled,” etc. to another element, there are no intervening elements present. Other words used to describe the relationship between elements should be interpreted in a like fashion (e.g., “between” versus “directly between,” “adjacent” versus “directly adjacent,” etc.). Similarly, a term such as “communicatively connected” includes all variations of information exchange and routing between two electronic devices, including intermediary devices, networks, etc., connected wirelessly or not.

As used herein, the singular forms “a,” “an,” and “the” are intended to include both the singular and plural forms, unless the language explicitly indicates otherwise. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof.

As used herein, “3D” means 3-dimensional, while “2D” means 2-dimensional. The structures and operations discussed below may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, to provide looping or other series of operations aside from single operations described below. It should be presumed that any embodiment or method having features and functionality described below, in any workable combination, falls within the scope of example embodiments.

The inventors have recognized that even well-trained surgical planners can struggle with limited information that is available in 2D surgical planning and/or without trying multiple approaches in planning prior to the operation. 3D virtual surgical planning may aid in determining the best plan and transferring it to reality. Particularly, surgery planning in a 3D view may be more accurate, realistic, and/or satisfying (to a surgeon as well as patient) as compared to a conventional process of 2D view-based planning. 3D planning, however, requires rendering of a 3D image from available data. The Inventors have recognized that X-ray images may be used for 3D reconstruction so that computational devices like mobiles phones or tablet computers, which have relatively lesser computational prowess, can also be used for the reconstruction process. Portability provided by such devices allows for greater flexibility in a healthcare environment. Hard copies of X-ray images of the region of the patient's body for operation, however, may not allow a surgeon to simulate post-operative conditions and/or may be an inconvenient way to perform measurements. Moreover, digital X-rays only provide 2D visualization of internal bone/joint anatomy and hence do not give accurate view, orientations, simulations, and/or feeling of surgery of a 3D environment.

A 3D surgical planning environment with 3D bone shapes may require a 3D virtual model of the bone. While such 3D models may be derived from CT scans of the bone anatomy of a patient, CT scans involve health risk, cost, and time, such that medical professionals may not prefer to perform surgery planning using CT scans. Moreover, 3D model reconstructions from CT scans are difficult on portable mobile devices, due to data size and computational requirements. Conversion of CT data to a 3D model is anyway time-consuming and requires significant manual inputs. Transferring CT scan data over the internet/network for various applications like tele-radiology, collaborative diagnosis, sharing, and saving a diagnosis or surgery planning, cloud-based medical applications based on 3D visualization of patients' anatomy may further be burdensome.

The Inventors have newly recognized that conversion of 2D X-ray images into 3D models may solve the above and other problems. Converting 2D X-ray images into 3D models may be computationally heavy and/or require X-ray images to be input in a way requiring a radiologist or surgeon to take extra care and/or use a special imaging device or a calibration device. In addition to the advantages of 3D surgical planning, 3D images/models of the bone can also be used for printing the bones into plastic models for informing patients about the surgery and/or training and real-model—based surgery planning. 3D models of bones can also be used for printing patient-specific instrumentation used in orthopedic surgeries. Use of 2D X-rays for 3D modelling does not require a patient to go under the health risk or expense of CT scanning. 2D imaging data is further much smaller and much more easily transferred than CT scan data for transfer to an instrumentation manufacturer. Thus, to overcome these newly-recognized problems as well as others and achieve these advantages, the inventors have developed example embodiments and methods described below to address these and other problems recognized by the Inventors with unique solutions enabled by example embodiments.

The present invention is devices, software as stored or executed on tangible computer-readable media, and methods for converting 2D X-rays into full 3D pre-operation planning models. In contrast to the present invention, the few example embodiments and example methods discussed below illustrate just a subset of the variety of different configurations that can be used as and/or in connection with the present invention.

The system and method of this invention relates to any elongate bone. p

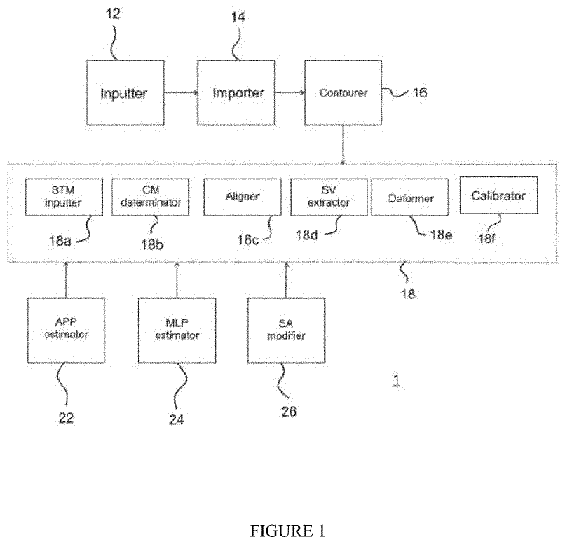

FIG. 1 is an illustration of a block diagram of an example embodiment system 1 useable to obtaining 3D images using conventional 2D X-ray images. For example, 3D models of bones may be generated from one or two 2D X-ray image/radiographs. Example embodiment system 1 is processor-based, and actions of system 1—and where example embodiment system 1 executes example methods—are dependent upon the processor(s) being specially-configured for the same. As shown in FIG. 1, an X-ray inputter 12 provides X-ray images for conversion. Inputter 12 may acquire the X-ray images through known procedures with conventional single-view X-ray imaging equipment. Orthogonal X-ray images from biplanar imaging may also be used. Such X-ray images from inputter 12 may include medial-lateral and anterior-posterior views. The X-ray images may not have any markers and/or have any known orientation with respect to the bone.

Alternatively, or additionally, a data importer 14 may import a patient's X-ray image(s) in digital format. For example, importer 14 may be a scanner configured to convert X-rays in hard copy format to a digitized format. This digitization may be done simply by using a camera, an X-ray digitizer, and/or an X-ray film scanner that converts the X-rays into digital format, such as any of the formats selected from JPG/TIF/PNG or DICOM format and the like. The X-ray images imported can belong to medial-lateral (ML) view or anterior-posterior (AP) view or both. Such imported images, may be processed for 3D reconstruction as final X-ray images in a digital format.

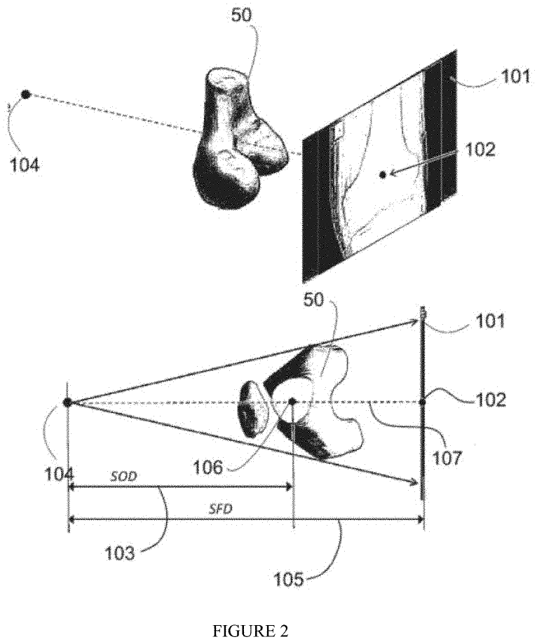

For 2D-to-3D conversion, a camera model determinator 18b may detect whether an X-ray image is ML or AP, using known parameters. As shown in FIG. 2, image plane 101 is a plane in a 3D imaging space that corresponds to detector plane 101, a plane coinciding with the flat X-ray sensor panel or a film of the real imaging environment, where the projection of the body/object/bone is formed. Image center 102 is the central position of a rectangular detector. For example, image center 102 may be the normal position on image plane 101, which coincides with the X-ray source, such as an X-ray sensor panel or a film is as placed during the imaging.

The determined camera model is used for 3D reconstruction to mimic the real X-ray imaging environment and includes the following: position of X-ray source 104, such as a point source corresponding to real X-ray source of the imaging equipment, with respect to image plane 101 in the imaging space; and the distance 103 between centroid 106 of an object such as bone 50 and the X-ray source 104, measured in the direction normal 107 to image plane 101 in the imaging space.

As shown in FIG. 2, for the camera model a position of source 104 with respect to image center 102, source film distance (SFD) 105, source object distance (SOD) 103 is defined. Position of the X-ray source 104 with respect to image center 102 is determined so that a normal of image plane 101 arising from image center 102 will coincide with source 104 at a known distance called source film distance 105 from image center 102. Typically, SFD 105 is equal to the distance between an X-ray source 104 and the detector, measured along the direction that is normal 107 to detector plane 101.

Source object distance 103 may be defined as the distance between X-ray source 104 and bone-centroid 106, which is the average position of all the surface points of bone 50, measured along direction normal 107 to image plane 101. A camera calibration perspective ratio K may be defined as a ratio of SOD 103 to SFD 105. SOD 103 may either be a known parameter or may be approximated. An example method to determine SOD 103 approximately is disclosed as below.

A spherical ball marker with a known actual diameter (for example, 25 mm) is placed near the object (bone 50/body) during X-ray imaging, closer to image center 102, at a height from detector plane 101, that is closer to the height of centroid 106 from detector plane 101, by eyeballing. SOD 103 will be equal to multiplication of SFD 105 and the ratio of the known actual diameter of the spherical ball marker to the diameter of the circular/elliptical projection of the spherical ball marker on detector plane 101. The diameter of the circular/elliptical projection of the spherical ball marker on detector plane 101 is equal to the diameter of the circular/elliptical projection of the spherical ball marker measured on the final X-ray image multiplied by the digital magnification ratio (given below).

A digital magnification ratio determinator for an X-ray image (ML or AP) may be included in example embodiments. The digital magnification ratio is the ratio of the value of the distance between the positions of the projections of any two points on the object's surface on detector plane 101 to the value of the distance between the corresponding points as measured in the final X-ray image, which may be measured in terms of pixels or mm. This ratio can be a known parameter, or an example method for determining the digital magnification ratio for an X-ray image may be used wherein a circular coin marker with known actual diameter is placed on the detector while taking the X-ray image. The digital magnification ratio will be approximately equal to the ratio of the known actual diameter of the circular coin to diameter of the coin as visible on the final X-ray image, as measured in terms of number of pixels or mm. All the positions determined on the final X-ray image, in terms of X and Y coordinates (e.g., in pixels) may be multiplied with the digital magnification ratio before processing for 3D reconstruction. This includes contour points and landmarks.

As shown in FIG. 1, example embodiment system 1 may include a contourer 16 that defines contours of a bone or other object in an uploaded or imported X-ray. The contour of bone is a curve consisting of set of 2D points on the final X-ray image which corresponds to the outer boundary of the bone that is visible on the final X-ray image. Contourer 16 may allow a user to draw an outer boundary of the bone anatomy of interest. Typically, a user draws the outer boundary of the bone anatomy of interest, depending on the surgery. For example, a femur and tibia bone for knee replacement or tibial osteotomy surgery may be outlined. Automated pre-defined contouring may be used to pre-empt contouring lines and assist the user in relatively more precise contouring. Brightness and/or contrast of the X-ray image may be adjusted so that the boundary of bone anatomy is easily distinguishable.

Contourer 16 may provide an initial contour for each bone that can be boundary of the projection of the template according to the calculated camera model. Since the vertices of the template will be divided and labelled as distinct regions, the projected initial contour will also have the distinction of the predetermined regions. A user may modify the initial contour to fit the bone's outer edge or boundary more precisely; the modification entails scaling, translation, rotation, deformation, etc. Contourer 16 may provide a touch interface wherein a user can touch a bone's boundary on the X-ray image and the contouring mechanism converts the touch interfaces to points, lines, and provides a continuous pattern in an intelligent manner. Defining contours using the contourer 16 is provided to define co-ordinates of the contour of the bone with respect to a relative or pre-defined center of an X-ray image. Typically, the X-ray in the medial-lateral plane is the x-z plane for the purposes of this invention. Typically, the X-ray in the anterior-posterior plane is the y-z plane for the purposes of this invention.

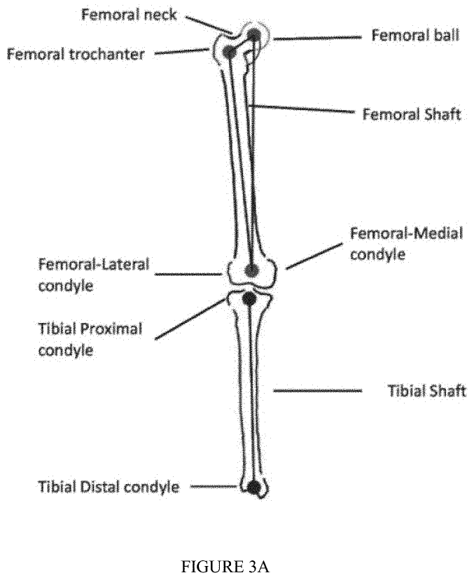

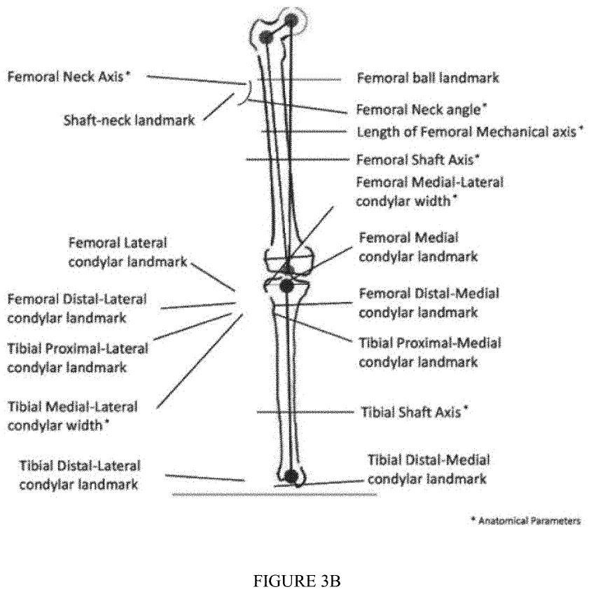

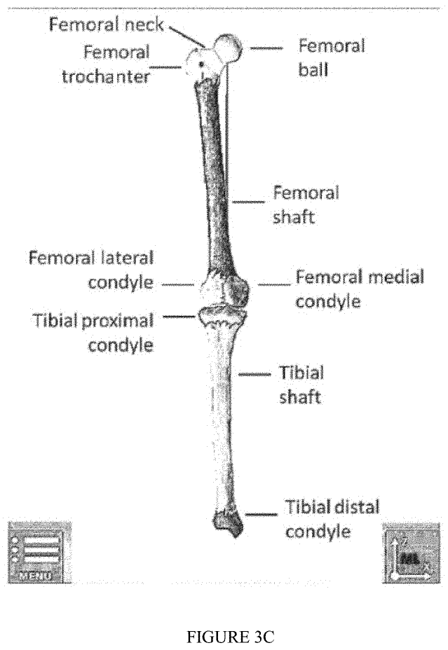

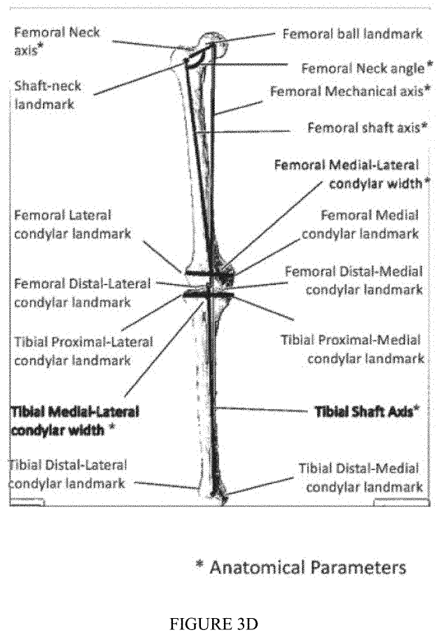

Anatomical regions may give anatomical landmarks to define anatomical parameters. Anatomical landmarks may be used for alignment of templates, and anatomical parameters may be used for selective anatomical modification of pre-created 3D templates. A 2D Anatomical Value may include: anatomical landmarks—2D positions of unique anatomical features identified on the final X-ray image on the basis anatomical regions; and anatomical parameters—values of geometric parameters like lengths and angles calculated based on anatomical landmarks to be used for 3D reconstruction. The points of the contour of bone may be divided into subsets in such a way that the subset points correspond to distinct anatomical regions of the bone. For a femur and tibia, FIG. 3A shows the anatomical regions.

For a femur bone, such as that shown in FIG. 3A, a contour of the bone in at least one view (ML or AP) of an X-ray image, the anatomical regions will be: femoral lateral condyle; femoral medial condyle; femoral shaft; femoral neck; femoral trochanter; and femoral ball. For a tibia bone such as that shown in FIG. 3A, a contour of the bone in at least one view (ML or AP) of X-ray image, the anatomical regions will be tibial proximal condyle, tibial shaft, and tibial distal condyle. The anatomical regions may be distinguished by drawing different regions of the contour in different colors if the contour is determined by drawing manually.

Based on the anatomical regions, anatomical axes are also determined manually or automatically. For a femur, the anatomical axis, shaft axis, and the neck axis may be determined. For a tibia, the anatomical axis and shaft axis may be determined. In a manual method of determination of any axis, a line may be fitted along user specified points that lie on the axis in the image. In another method of determination of any axis, a user may place a given line or curve (in case of shaft axis) along the position and orientation of the required axis. In an automatic method, a geometric calculation is performed on the distinguished anatomical regions of the contour. For example, a best fit line to the femoral shaft region of the contour may be assigned as the femoral anatomical axis. Or, for example, a best fit Bezier curve to the femoral shaft region of the contour may be assigned as the femoral shaft axis. Or, for example, a best fit line to the femoral neck region of the contour may be assigned as the femoral neck axis. Or, for example, a best fit line to the tibial shaft region of the contour may be assigned as the tibial anatomical axis.

Positions of anatomical landmarks may be determined on the final X-ray image with respect to the extracted contours, based on anatomical regions. For a femur and tibia, FIG. 3B shows the anatomical landmarks and the anatomical parameters. In a manual method of determination of anatomical landmarks, a user may specify points on the image that lie on the landmark. In an automatic method of determination of anatomical landmarks, the anatomical landmarks, as mentioned above, may be determined from the final X-ray image by calculating geometric features, such as extreme position in a direction, or a centroid, or a peak, of the above-mentioned anatomical regions of the contour maybe with respect to some known anatomical axes.

As shown in FIG. 3B, for a femur, the following landmarks were identified, on an AP view X-ray image: Femoral Distal-Lateral condylar landmark—a position of the extreme distal point along the Femoral anatomical axis of the Femoral lateral condyle region of the contour; Femoral Distal-Medial condylar landmark—a position of the extreme distal point along the Femoral anatomical axis, of the Femoral medial condyle region of the contour; Femoral Lateral condylar landmark—a position of the extreme lateral point along the line passing through the Femoral Distal-Lateral condylar landmark and the Femoral Distal-Medial condylar landmark; Femoral Medial condylar landmark—a position of the extreme medial point along the line passing through the Femoral Distal-Lateral condylar landmark and Femoral Distal-Medial condylar landmark; Femoral ball landmark—an average of the position of the center of the best fit sphere to all the points of the femoral ball region of the contour; Greater Trochanter tip landmark—a position of the extreme proximal point of the Femoral trochanter region of the contour; and Shaft-Neck landmark—a position of the intersection of the femoral anatomical axis and the AP femoral neck axis.

For a femur, the following landmarks were identified, on an ML view X-ray image: Femoral Distal-Lateral condylar landmark—a position of the extreme distal point along the Femoral anatomical axis, of the Femoral lateral condyle region of the contour; Femoral Distal-Medial condylar landmark—a position of the extreme distal point along the Femoral anatomical axis, of the Femoral medial condyle region of the contour; Femoral Posterior-Lateral condylar landmark—a position of the extreme posterior point perpendicular to the direction of femoral anatomical axis, of the Femoral lateral condyle region of the contour; Femoral Posterior-Medial condylar landmark—a position of the extreme posterior point perpendicular to the direction of femoral anatomical axis of the Femoral medial condyle region of the contour; Femoral Anterior-Lateral condylar landmark—a position of the extreme anterior point perpendicular to the direction of femoral anatomical axis of the Femoral lateral condyle region of the contour; Femoral Anterior-Medial condylar landmark—a position of the extreme anterior point perpendicular to the direction of femoral anatomical axis, of the Femoral medial condyle region of the contour; Femoral ball landmark—an average of the position of the center of the best fit sphere to all the points of the femoral ball region of the contour; and Greater Trochanter tip landmark—a position of the extreme proximal point of the Femoral trochanter region of the contour.

For a tibia, the following landmarks were identified, on an AP view X-ray image: Tibial Proximal-Lateral condylar landmark—a position of the Extreme lateral point perpendicular to the direction of tibial anatomical axis, of the tibial proximal condyle region of the contour; Tibial Proximal-Medial condylar landmark—a position of the Extreme medial point perpendicular to the direction of tibial anatomical axis, of the tibial proximal condyle region of the contour; Tibial Distal-Lateral condylar landmark—position of the Extreme lateral point perpendicular to the direction of tibial anatomical axis, of the tibial distal condyle region of the contour; and Tibial Distal-Medial condylar landmark—position of the Extreme medial point perpendicular to the direction of tibial anatomical axis, of the tibial distal condyle region of the contour.

For a tibia, the following landmarks were identified, on an ML view X-ray image: Tibial Proximal-Posterior condylar landmark—a position of the Extreme posterior point perpendicular to the direction of tibial anatomical axis, of the tibial proximal condyle region of the contour; Tibial Proximal-Anterior condylar landmark—a position of the Extreme anterior point perpendicular to the direction of tibial anatomical axis, of the tibial proximal condyle region of the contour; Tibial Distal-Posterior condylar landmark—a position of the Extreme posterior point perpendicular to the direction of tibial anatomical axis, of the tibial distal condyle region of the contour; and Tibial Distal-Anterior condylar landmark—a position of the Extreme anterior point perpendicular to the direction of tibial anatomical axis of the tibial distal condyle region of the contour.

Anatomical Parameters may be calculated automatically based on anatomical landmarks; parameters can be a distance between two landmarks, an angle between lines defined by any two landmarks, and/or any correlative value between landmarks. For a femur, on AP X-ray image, the following parameters were identified: Femoral Medial-Lateral condylar width—the distance between femoral Lateral condylar landmark and femoral Medial condylar landmark; Femoral Shaft length—the distance between femoral shaft-neck landmark and a position of intersection of femoral AP anatomical axis and a line connecting Femoral Distal-Lateral condylar landmark and Femoral Distal-Medial condylar landmark; Length of Femoral Mechanical axis—the distance between femoral ball landmark and the center of Femoral Distal-Lateral condylar landmark and Femoral Distal-Medial condylar landmark; Femoral Neck length—the distance between AP Femoral ball landmark and Shaft-Neck landmark; and Femoral Neck angle—the angle between AP femoral anatomical axis and AP femoral neck axis.

For a tibia, on AP X-ray image, the following parameters were identified: Tibial Medial-Lateral condylar width—the distance between tibial Proximal-Lateral condylar landmark and tibial Proximal-Medial condylar landmark; and Tibial Shaft length—the distance between a position of intersection of tibial AP anatomical axis and a line connecting tibial Proximal-Lateral condylar landmark and tibial Proximal-Medial condylar landmark and a position of intersection of tibial AP anatomical axis and a line connecting tibial Distal-Lateral condylar landmark and tibial Distal-Medial condylar landmark.

In example system 1 for converting 2D to 3D surgical data, bone template model inputter 18 a may provide a corresponding bone template model in 3-dimensional format. The corresponding bone template model format may be a clinically normal bone in the form of 3D mesh with triangular elements. This bone template model may be reconfigured into a shape that matches the input contours as defined by contourer 16. The pre-created 3D template may be formed in the form of mesh, pre-created from a CT scan of some healthy/average subject or subject with matching medical condition to a patient whose input X-ray images are used for the 3D reconstruction. A data set with multiple subjects may be created. Demographics and gender of subjects may be used to make discreet the data set. Different template shapes belonging to different ages or age groups, ethnicity groups, etc. may be created and stored.

A 3D surface model can be created using techniques such as MIMICs through segmentation of all the slices images of CT scan. The surface model can be exported as point cloud surface model. A point cloud is a set of data points in some coordinate system. In a 3D coordinate system, these points are usually defined by X, Y, and Z coordinates and are often intended to represent the external surface of an object (such as bone 50). Connectivity between points of the point cloud can be formed using methods like constrained Delaunay Triangulation to form a 3D mesh model with triangular elements. A triangular element is an element which is defined by forming connectivity between three points. By triangulation of all the points of the point cloud a mesh of triangular element may be formed. The point cloud may be sampled to reduce the number of surface points, and hence the number of triangular elements resulting from meshing. Depending on extent of sampling, or point cloud density, sampling related parameters, such as reduction in volume formed by the closed mesh, may be defined to form an optimum model such that errors are minimum and bone shape features are preserved, but points are relatively reduced.

A surface model may be exported from a dense cloud—for example, a cloud with 1 mm point-to-point mech distance. The surface model may then be uniformly sampled to a sufficient number of points. A sufficient number of points may be determined by measuring the level of detail of the 3D bone model. The level of detail and the volume (of the closed meshed model) gets reduced after the sampling. The reduction in level of detail can be determined by measuring the difference in volume of a closed mesh created from the initial dense point cloud and that of a closed mesh created from the sampled points. By putting the threshold on the level of detail, such as a volume reduction of 2%, the sampling and sufficient number of points may be determined. The point-to-point distance at this condition, in an example of a femur bone template, may be 3 mm. A 3D mesh with triangular elements may be created from the sampled points and used as the template model for the 3D reconstruction. The template model may be in the form of triangular surface mesh with sets of a number of vertices and a number of faces. For a truncated distal femur bone template, the number of vertices may be 1795 and the number of faces may be 3559, for example. These example numbers of points are sufficient to define the distal femur part of the bone with its shape features.

The anatomical regions, anatomical axes, anatomical landmarks, and anatomical parameters of the 3D template model may be pre-determined, at least manually.

In at least another embodiment, an improved 3D reconstruction system and method is provided for deformed elongate bones. This system and method requires four input items:

Calibration parameters obtained from a calibrator 18f;

Contour points, obtained from the contourer 16, from 2D X-ray images taken in approximate AP and approximate ML view;

Landmark points, obtained using camera model determinator 16, from 2D X-ray images taken in approximate AP and approximate ML view; and

3D bone template, obtained from the bone template model inputter 12

Calibration parameters include position of X-ray source: S(x, y, z), Source Film distance: SFD, principal point position: PPos (X, Y), vectors dirX and dirY representing orientation of the image plane in 3D space. The calibration parameters for AP and ML images are in the same 3D space since a single calibration marker object is used while taking both images. This 3D imaging space is transformed such that the vectors dirX and -dirY for AP image align with XZ plane. Using the calibration parameters, the X-ray imaging can be simulated for 3D reconstruction.

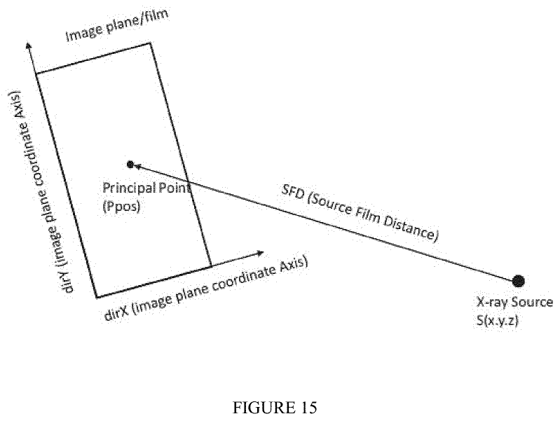

FIG. 15 shows the imaging space with calibration parameters.

Contour points Cn (X, Y) and landmark points Lk (X, Y), for a bone, are in 2D coordinate system as they are extracted, by a user, from a 2D X-ray image. The landmark points Lk include all essential landmarks of the bone to represent its deformity. The following Table 1 lists down some example anatomical landmarks.

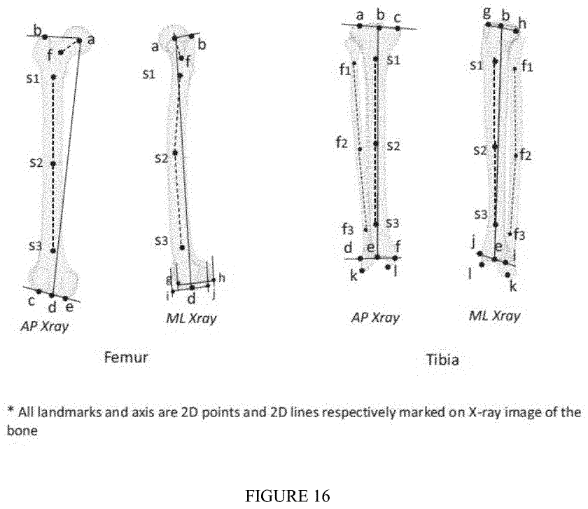

FIG. 16 depicts the 2D anatomical landmarks and 2D anatomical axes on a bone X-ray.

TABLE 1
Anatomical Landmarks and Anatomical Axes (Joint line, mechanical)
extracted from lower limb AP and ML X-ray images

| Femur (AP-ML landmarks | Tibia (AP-ML landmarks |
|---|---|
| and Axes) | and Axes) |
| 2D Points | 2D Points |
| Ball centre | Proximal lateral condyle extreme |
| Trochanter tip | Proximal jointline centre |
| Distal lateral condyle extreme | Distal lateral condyle extreme |
| Distal jointline centre | Distal lateral condyle extreme |
| Distal medial condyle extreme | Distal jointline centre |
| Neck centre | Distal medial condyle extreme |
| | Proximal anterior condyle extreme |
| | Proximal posterior condyle extreme |
| | Distal anterior condyle extreme |
| | Distal posterior condyle extreme |
| | Lateral malleolus tip |
| | Medial malleolus tip |
| 2D Lines and curves | 2D Lines and curves |
| ab) Proximal jointline | abc) Proximal jointline |
| cde) Distal jointline | def) Distal jointline |
| ad) Mechanical axis | be) Mechanical axis |
| s1s2s3) Shaftpoints curve | s1s2s3) Shaftpoints curve |
| gh) Medial anterior-posterior | f1f2) Proximal fibular axis |
| jointline | |
| ij) Lateral anterior-posterior | f2f3) Distal fibular axis |
| jointline | |

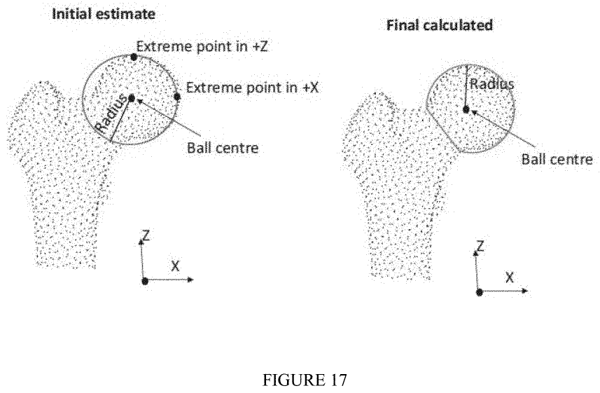

FIG. 17 illustrates computation of landmark based on the standard directions of a bone's anatomical co-ordinate system.

The contour points Cn are the boundary of those selected regions of lower limb bones which represents torsional deformity and knee joint deformity. The regions include distal femur and proximal tibia in both AP and ML X-ray image. The distal femur contour is divided into 3 parts i.e. condylar shaft, medial condyle, and lateral condyle. The proximal tibial contour is a single condyle part.

The template is a 3D model of a bone in the form of mesh (object with vertices and faces) with triangular elements. The vertices of this mesh represent the points on the surface of the bone (femur or tibia). This template is modified through various steps into a final required 3D bone model. For the template to be suitable for 3D reconstruction, it has to undergo certain preparation steps which include: segmentation of template into various anatomical regions, identification of anatomical landmarks of template, and determination of Anatomical Coordinate System (ACS). These preparation steps as explained below, in terms of processes defined as Template Segmentation Process, Landmark Identification Process, and Anatomical Coordinate System.

The template segmentation process is defined by at least the following steps:

Step 1: Calculating the three principal axes by applying principal component analysis (PCA) on the vertices 3D position data of the template.

Step 2: Rotating the template in such a way that first principal axes matches with Z axis, second principal axes with X axis, and third principal axes with Y axis respectively. In this position, the standard coordinate axis can be considered as an initial estimate of ACS for the template bone.

Step 3: Separating the middle ⅗th vertices of the template bone along Z axis and finding the best-fit cylindrical axis to these vertices region.

Step 4: Realigning the template such that the best-fit cylindrical axis becomes parallel to Z axis

Step 5: Extracting the bottom ⅕th vertices and top ⅕th vertices of the template along Z axis as distal and proximal segment (storing indices) respectively. The rest of the vertices are shaft segment.

For femur, there are few more additional steps:

Step 6: The distal condyle segment is divided into three sub-segments namely medial condyle, lateral condyle and condylar shaft. The top half is condylar shaft while bottom half is divided further into right and left half as medial and lateral condyle respectively (for right bone template).

Step 7: The proximal condyle segment is divided into four sub-segments namely femoral ball, femoral neck, femoral greater trochanter and femoral lesser trochanter. From the proximal segment, the extreme points along positive Z axis and positive X axis is calculated and an initial estimate of femoral ball sphere radius and centre is calculated (FIG. 17). Vertices included in a spherical region with 1.1 times the estimated radius around the estimated centre are extracted. Best-fit sphere radius and centre is determined for the extracted region. The vertices with the coefficient of determination (R2) below a threshold are extracted as the femoral ball segment. The threshold is the value at which the R2 suddenly jumps.

Step 8: The remaining region tangent to sphere is extracted as neck, greater trochanter and lesser trochanter

FIG. 3C illustrates these anatomical regions as corresponding to the regions distinguished in the contour of an X-ray image. Anatomical landmarks identified based on anatomical regions of the template may be the same as the anatomical landmarks identified based on anatomical regions of the contour, as shown in FIG. 3D. Anatomical parameters identified based on anatomical landmarks of the template may be the same as the anatomical parameters identified based on anatomical landmarks of the contour.

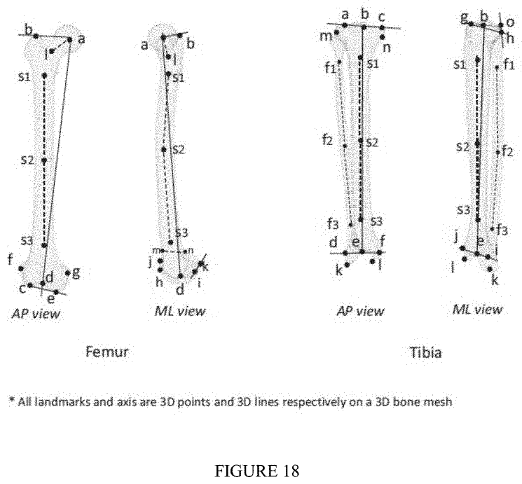

In the landmark identification process, landmarks are calculated based on the standard directions of bone's ACS. In the given ACS (initially estimated above), the following 3D landmarks are calculated. (FIG. 18)

TABLE 2
Landmarks and Axes (Joint line, mechanical)
calculated in 3D vertices of bone

| Femur (AP-ML landmarks | Tibia (AP-ML landmarks |
|---|---|
| and Axes) | and Axes) |
| 3D Points | 3D Points |
| Ball centre | Proximal lateral condyle extreme |
| b) Trochanter tip | Proximal jointline centre |
| c) Distal lateral condyle extreme | Distal lateral condyle extreme |
| d) Distal jointline centre | Distal lateral condyle extreme |
| e) Distal medial condyle extreme | Distal jointline centre |
| f) Lateral condyle extreme | Distal medial condyle extreme |
| g) Medial condyle extreme | Medial Anterior condyle extreme |
| h) Anterior medial condyle extreme | Medial posterior condyle extreme |
| i) Posterior medial condyle extreme | Distal anterior condyle extreme |
| j) Anterior lateral condyle extreme | Distal posterior condyle extreme |
| k) Posterior lateral condyle extreme | Lateral malleolus tip |
| l) Neck centre | Medial malleolus tip |
| | Lateral knee condyle extreme |
| | Medial knee condyle extreme |
| | o) Lateral posterior condyle |
| | extreme |
| 3D Lines and curves | 3D Lines and curves |
| ab) Proximal jointline | abc) Proximal jointline |
| cde) Distal jointline | def) Distal jointline |
| ad) Mechanical axis | be) Mechanical axis |
| ik) Posterior-condylar axis | ho) Posterior-condylar axis |
| s1s2s3) Shaftpoints curve | s1s2s3) Shaftpoints curve |
| al) Neck axis | f1f2) Proximal fibular axis |
| mn) Anterior-posterior | f2f3) Distal fibular axis |
| jointline | |
| | gh) Anterior-posterior proximal |
| | jointline |
| | ij) Anterior-posterior distal |
| | jjointline |

The anatomical coordinate system (ACS) (FIG. 23) defining process is further explained, below.

The anatomical parameters defining the deformity in a bone are calculated along standard directions like Anterior-Posterior (AP) and Medial-Lateral (ML) and Superior-Inferior (SI). E.g. torsional deformity is measured as angle between femoral ball neck axis and posterior condylar axis, along the SI direction. A condylar deformity is measured as an angle between mechanical axis and distal joint line along both AP and ML direction separately. These three directions constitute the ACS for a bone and can be calculated based on the anatomical landmarks. The Z-axis (SI direction) of the ACS is along the mechanical axis of the bone. The Y-axis (AP direction) of the ACS is along the cross-product of the mechanical axis and the posterior-condylar axis. The X-axis (ML direction) of the ACS is the cross-product of the Y-axis and the Z-axis of the ACS. The landmarks can be re-calculated based the new ACS and vice-versa iteratively.

FIG. 18 illustrates computed anatomical landmarks.

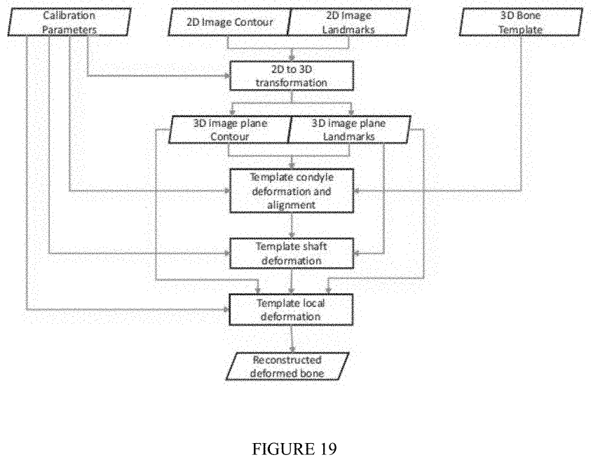

FIG. 19 illustrates the 3D reconstruction flowchart.

First the contour and landmark points in 2D coordinate system are transformed into 3D coordinate system (Cn (x, y, z) and Lk (x, y, z)) of the imaging space. This is done using the calibration parameters for both AP and ML X-ray images. This is followed by the alignment of the template in the 3D imaging space. The template is then deformed at various regions to reconstruct the deformity in input bone. This includes shaft bending deformity, condyle region deformity and torsional deformity. Finally, the bone template is deformed in such a way that its projection on the X-ray image plane in the imaging space will match exactly with the input contour. The above mentioned method is explained below:

2D to 3D coordinate system transformation:

Principal point PPos(x, y, z) is defined as a 3D point at the distance SFD from the source in the direction normal to dirX and dirY. The transformation parameters are calculated in such a way that PPos(X, Y) transforms to PPos(x, y, z), X and Y coordinate axis of the image coordinate system aligns with dirX and —dirY vector respectively. Using this transformation, Cn(X, Y) and Lk(X, Y) is transformed into Cn(x, y, z) and Lk(x, y, z) respectively. Cn(x, y, z) and Lk(x, y, z) of AP and ML images are in same 3D space because respective calibration parameters are also in same 3D space.

Initial Template alignment and condyle deformation:

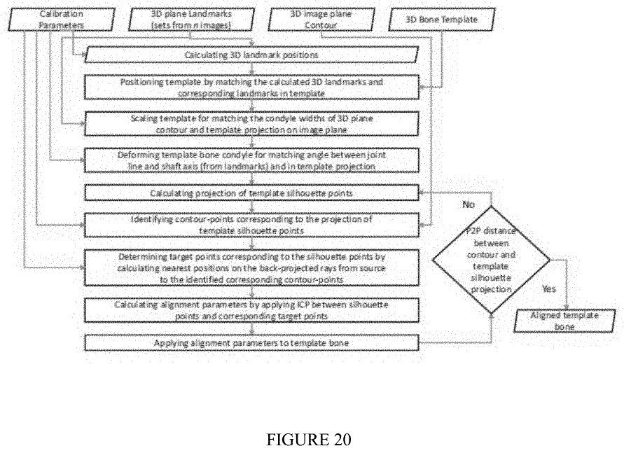

(FIG. 20 illustrates a flowchart for initial template alignment and condyle deformation.) This involve iterations between three steps:

Scaling of the template such that width of projection of the distal condyle segment on the image plane along the joint line matches the same calculated from the landmarks Lk;

Non-rigid deformation (Laplacian surface deformation) of distal condyle segment in such a way that the angle between anatomical axis and joint line in its projection matches the angle calculated from the landmarks Lk (Fig); and

3D transformation of the template in such a way that the boundary of the projection of its condyle segment (distal for femur and proximal condyle for tibia) onto the image plane best-fits with the respective contour Cn, for both AP and ML.

The second step is performed using ICP (Iterative closest point) based method. However, in the current method, of this invention, 3D point-pairs required for the ICP, are calculated separately for medial condyle, lateral condyle and condylar shaft. This results in better 3D alignment compared to when the 3D point-pairs are calculated for the whole condyle altogether (especially in the ML view). This is because the separate point-pairs result in the accurate relative positioning of medial and lateral condyle which in-turn results in accurate alignment along the shaft axis.

The iteration stops when the average point-to-point distance between the boundary of the projection and the respective contour do not change. This usually happens in two to three iterations. This iterative process results in accurate condyle deformation as well as accurate alignment of the template in 3D space. The rest of the template segments are deformed in further steps.

Template shaft deformation:

This process is required to reconstruct the bending and torsional deformity in the bone.

This involves three steps:

Building 3D reference shaft axis from input shaft axis landmarks,

Deformation of template shaft segment to match the anatomical axis,

Twisting of template shaft segment

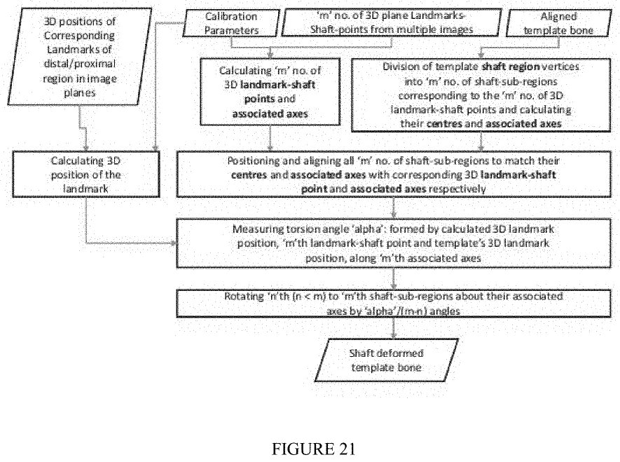

The shaft axis landmarks in AP and ML images have end to end correspondence throughout from its distal to proximal end and are equal in numbers (m). Hence using the calibration parameters and the shaft axis landmarks in both AP and ML image, a 3D shaft axis (further referred as reference shaft axis) can be calculated in form of set of m-l number of 3D line segments as shown in schematic FIG. 21 (FIG. 21 illustrates a flowchart for deforming a template bone).

The template shaft deformation includes calculation of template shaft axis and deforming the shaft segment such the template shaft axis matches with the reference shaft axis. The template shaft segment is divided into 10 sub-segments along its first principal axis. The centroids of vertices belonging to the open boundary of shaft segment mesh are calculated at its distal and proximal ends (boundary-centroids). The template shaft axis is calculated as a Bezier curve with m number of uniform points passing through centroid of each sub-segment and the boundary-centroids. The shaft segment vertices are re-divided into m-l number of sub-segments based on their positions with respect to the m-l line segments of template shaft axis. Affine transformations are calculated for each m-l line segments of the template shaft axis so that they coincide with corresponding m-lline segments of the reference shaft axis. The same transformations are applied to the associated shaft sub-segments. The m number should be sufficiently large to get a smoothly deformed (bending deformity) shaft segment.

Using the calibration parameters and the femoral ball-centre (medial malleolus in case of tibia) landmarks in both AP and ML image, a reference femoral ball centre is calculated. The position of the reference femoral ball centre and reference femoral neck axis with respect to the reference shaft axis represents the torsion in the bone. To match the position of the template femoral ball centre to the position of reference ball centre, the template shaft sub-segments are twisted. This is done in a distributed way by appropriate rotation of each m-l shaft sub-segments about their associated line segments of the template shaft axis. This brings the accurate torsion in the shaft but the template femoral ball centre will not exactly match the reference femoral ball centre and template femoral neck axis will not exactly match the reference femoral neck axis. This final matching of the femoral ball centre position, neck axis and trochanter position is done by non-rigid deformation of proximal femoral region. This is done using Laplacian surface deformation explained in next step. This results in the accurate reconstruction of femoral ball and neck region.

For tibia, the process is same except that the matching of both the malleolus position, proximal fibular axis and distal fibular axis is considered in place of femoral ball centre position, trochanter position and femoral neck axis.

Local Deformation:

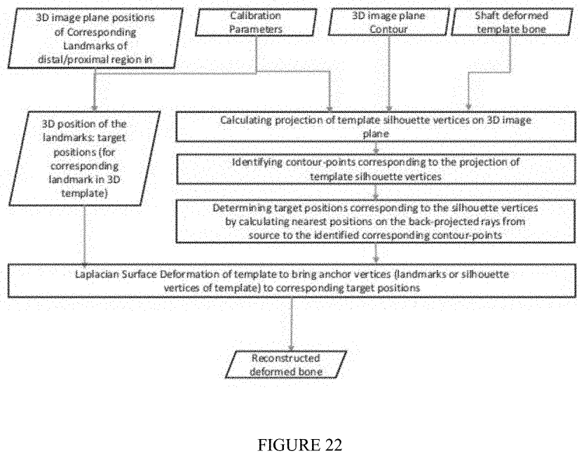

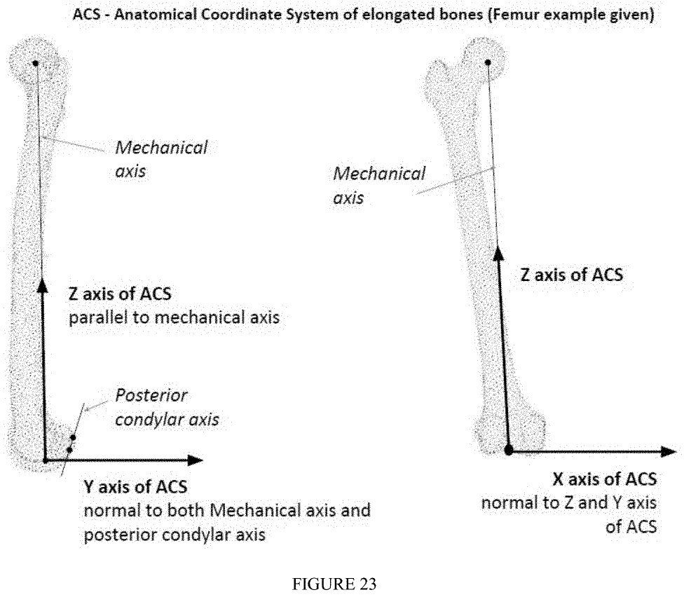

(FIG. 22 illustrates a flowchart for local deformation.)

The local deformation results in an accurate surface reconstruction of the bone. However, the above mentioned process globally reconfigures the template to reconstruct the bone anatomical deformity in the template and to make it suitable for local deformation. The local deformation may be performed using Laplacian surface deformation. Laplacian surface deformation smoothly deforms a mesh while bringing a few selected anchor points of the mesh to respective target positions (positional constraints) and maintaining the inter-vertices positional relationship (Laplacian constraints) described by Laplacian coordinates. A least square method is applied to follow both positional and laplacian constraints. The anchor points are silhouette points of the template bone for AP and ML view calculated using respective X-ray source positions and image planes. The silhouette points are projected on the image planes and their corresponding contour points are identified based on Self organizing maps as explained in et.al. From each corresponding contour point, a ray is back projected to the source and a nearest position on the ray from each silhouette point is determined. This nearest position is the target position for the silhouette point (anchor).

In example embodiment system 1, a 2D-to-3D converter 18 converts the 2D X-ray images to 3D images. The conversion may be based on Laplacian deformation, which is an efficient shape deformation technique. The generated 3-dimensional model may a surface model and/or a solid model, with the surface model having reduced computational requirements. A silhouette vertices extractor 18d in converter 19 may extract silhouette vertices and projections of a 3-dimensional template, at its aligned position, in accordance with the determined camera model, using known parameters. Silhouette vertices are those vertices of the template which form the outer contour of the template's projection on image plane 101, according to camera model, hereinafter called a template projection contour.

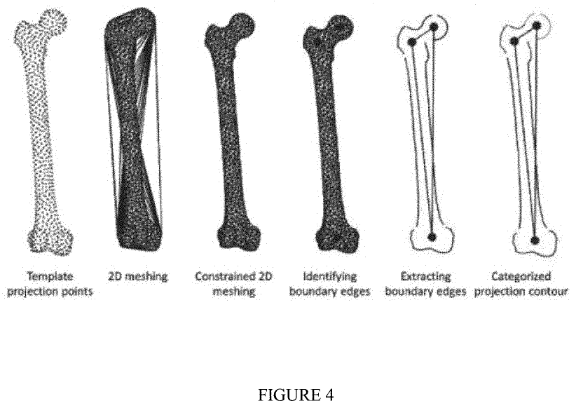

For a camera model, a perspective projection of the vertices of the template mesh may be computed on its image plane. The outer contour of the template projection, or template projection contour, can be computed using the following example method. All vertices of the template may be projected on image plane 101 (perspective projection). Triangulation meshing of projection is obtained by using Delaunay triangulation method (2DM). Using constraint Delaunay triangulation method, a 2D mesh (2CDM) with triangular elements is created from the projected points as seen in FIG. 4, illustrating triangulation of projected points, meshing after putting constraints and the outer contour calculation. Those edges of the triangular elements which are shared with only one triangular element are the boundary edges and the corresponding projected points are the boundary point and hence the template projection contour points. The silhouette vertices are those vertices of the template which form the outer contour of the template's projection (template projection contour) on image plane 101, according to a camera model.

An example embodiment 2D-to-3D converter 18 may include an aligner 18c that aligns a pre-created 3-dimensional template of a bone with respect to the contour points. The pre-created 3-dimensional template may be formed in a mesh, pre-created from a CT scan of some clinically normal bone, such as from a data set with multiple subjects. Alignment of the pre-created 3-dimensional template differs according to the image view and bone anatomy. For example, the image view may be one from medial-lateral or one from anterior-posterior.

Alignment may be performed in the context of a femur bone, for example. Converter 18 may include anterior-posterior pose estimator 22 configured to determine a first alignment of a femoral template with respect to the anterior-posterior input X-ray image. Input to estimator 22 may be taken from the contourer 16, which has contoured data and image of a bone's X-ray in its anterior-posterior view. A joint center may be located, and the template projected on to an image plane with arbitrary initial positions and orientation. This assists in deformation of the femoral template for 3D reconstruction. The template models (femur and patella), obtained from the bone template model inputter 12 may be in the form of surface point cloud.

A source-film distance 105 is calculated, and a source-object distance 103 is calculated. The projection may be determined as perspective type and calculated according to a camera model. Then an automatic initialization may place the contour points on image plane 101 of the camera model. The template may be positioned and/or translated between X-ray source 104 and image plane 101 of the camera model, in such a way that the template's centroid 106 is at the distance of SOD 103 from the X-ray source 104, measured along a normal 107 to image plane 101. Centroid 106 may be defined as the average of the positions (x,y,z) of the vertices of the template. Orientation of the template may make image plane 101 parallel to that plane of the template (ML or AP) of which the contour belongs to. The template may be rotated about the normal to image plane 101 passing through the template's centroid 106, in such a way that the projection of its anatomical axis (by the camera model) becomes parallel with the anatomical axis of the contour. The templates may be translated along directions parallel to image plane 101 in such a way that centroid 106 of the bone template projection coincides with that of the contour.

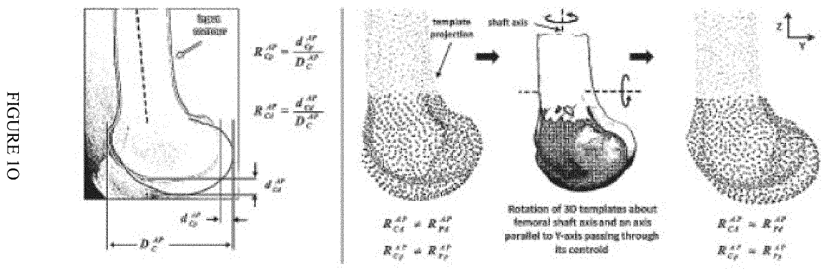

As shown in FIG. 9, after automatic initialization, a two-step procedure may be applied to find the template's pose in 3D. A patellar template may be rigidly translated or rotated with the femoral template. In “Step 1,” the templates (femur and patella) are rotated about an anatomical axis, e.g., parallel to Z-axis, to match the position of the joint center with respect to the template in its projection on image plane 101 with that in the input contour. The horizontal distance, measured along a direction perpendicular to anatomical axis and a normal to image plane, “dcml” between the joint center and the anatomical axis is calculated from the input contour. The ratio “rcml” of distance “dcml” to medial-lateral width “dcml”—distance between femoral Lateral condylar peak and femoral Medial condylar peak—of the femur bone is also calculated from the input contour. Similarly, distance “dpml” and ratio “rpml” are calculated from the femoral template projection. Finally, the templates are rotated about the anatomical axis such that the ratio “rpml” matches the ratio “rcml.”

If the distance “dcml” is constant, an angle of rotation about the anatomical axis can be calculated using the relation between the distance “dcml” and patellar angle as shown in FIG. 9. After rotation about the anatomical axis, distance, and hence ratio, changes. Hence, the process is applied iteratively until the difference rpml-rcml becomes very small.

To locate a joint center, on the contour (ML view), the joint center is the position of the centroid of the points of the contour of patella bone visible on the X-ray image. On the template projection, the joint center is the position of the centroid of the points of projection of the template of Patella bone, which is always rigidly positioned with respect to the femur bone template. In case the femur bone is truncated, after step 1, the input contour and the template projection are first processed for the equivalence in shapes. The input contour of the bone was truncated to match its aspect ratio to that of the projection. Also, the outer boundary of the femoral template projection (projection contour) is extracted automatically using the silhouette vertices' extraction.

As shown in FIG. 9, in Step 2, the extracted femoral template projection contour is aligned to the input contour using a shape registration method like iterative closet point analysis (ICP). Optimal values transformations (translation, scaling, and rotation) are calculated using ICP, for the template projection contour to align it with the input contour. Corresponding transformations (translation, scaling, and rotation) are applied to the template in such a way that its projection on image plane 101 (after applying transformations) will match with the aligned template projection contour.

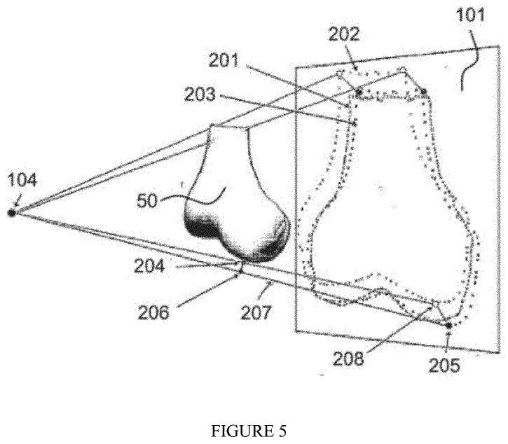

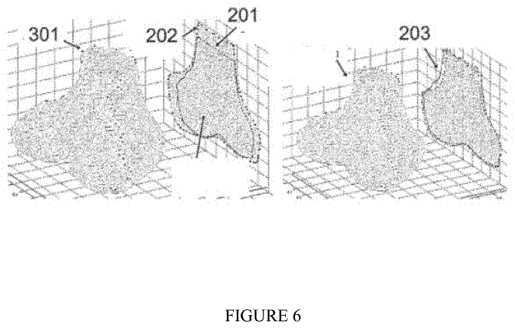

As shown in FIG. 5, to apply corresponding transformations to the template, 3D-3D point pairs are determined after the final alignment of template projection with the contour points of anterior-posterior view. This may be performed using a back projection method. Input contour 201 is provided by a user using an X-ray image. Further, template projection contour 202 that is input using the system and method of this invention, which template projection contour is provided before alignment. Aligned template projection contour 203 may be provided after alignment of the template projection with respect to the input contour defined by the user. For each number of silhouette vertices, a silhouette vertex of the template with its initial position asₘ 204 corresponding to the template projection contour point ppₘb 205, a closest position bsₘ 206 on the projection ray rₘ 207 joining the X-ray point source 104 and the corresponding aligned template projection point ppm 208 is calculated using a template projection point ppₘb 205 available before alignment. In this way, total M numbers of 3D-3D point pairs (asₘ, bsₘ) are found for each silhouette vertex. ICP technique was applied on these point pairs (asₘ 204, bsₘ 206) to find the transformations of silhouette vertices 301 for their optimal superimposition and applied to the whole template model. FIG. 6 shows the template model before and after the alignment.

In the iterative process of the ICP method, after each step of the iteration, a new corresponding points' pair between template projection and input contour may be determined. After each step of the iteration, the mean absolute distance (MAD) between the points of template projection contour and their corresponding closest points of the input contour may be measured. The iteration is stopped when the difference in MAD of the two consecutive steps of iterations is below 0.0001 mm. The MAD between the input contour and the template projection contour is minimized through the iteration. The corresponding alignment of the 3D template is then applied at once.

Example embodiment system 1 may include a medial-lateral pose estimator 24 configured to determine a second alignment of the template with respect to the input X-ray image, for a femur bone shape. Input to estimator 24 may be taken from contourer 16 which has contoured data and image of a bone's X-ray in its anterior-posterior view. An anterior-posterior projector projects the anterior-posterior image on to an image plane with arbitrary initial positions and orientation. This assists in formation of template models. The template model of femur, obtained from the bone template model input mechanism, is in the form of surface point cloud.

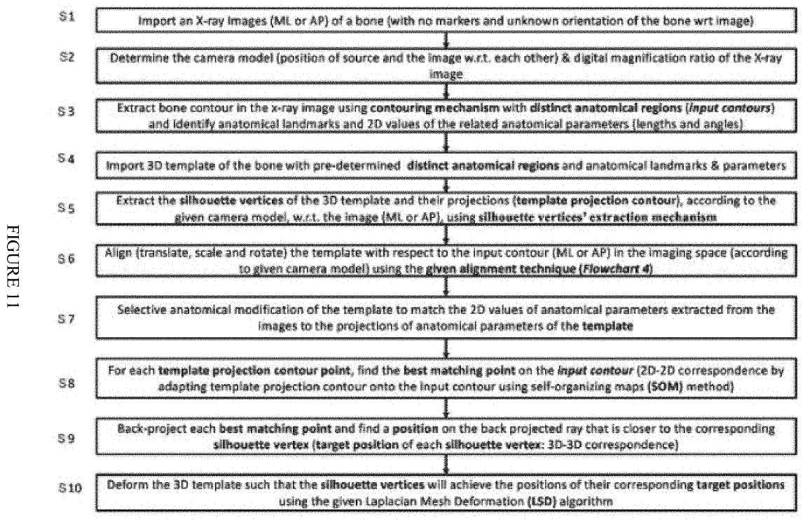

As shown in FIGS. 9 and 10, from the ML view X-ray image, separate boundary contours may be manually extracted for bone shaft, medial bone side, and lateral bone side. FIG. 9 illustrates template alignment with respect to Anterior-Posterior image and FIG. 10 illustrates template alignment with respect to Medial-Lateral image. The automatic initialization process may be similar as that for the anterior-posterior view. After the initialization, the two-step procedure is applied.

The template is first rotated about the shaft axis. For this, a ratio “rcapd” of distance between Posterior-Lateral condylar peak and Posterior-Medial condylar peak of the bone to the anterior-posterior width, both measured along direction perpendicular to anatomical axis and a normal to image plane, may be calculated from the contour in FIG. 10. Similar ratio “rpapd” may be calculated from the template projection on image plane. The template is rotated about the anatomical axis so that the ratio “rpapd” matches with the ratio “rcapd.” The angle of rotation may be calculated using a trigonometric function.

The template is then rotated about an axis that is direction perpendicular to anatomical axis and a normal to image plane and passing through its centroid. To calculate the angle of rotation, a ratio “rcapp” of distance between Femoral Distal-Medial condylar landmark and Femoral Distal-Lateral condylar landmark (measured along the anatomical axis) to the anterior-posterior width (measured along a direction perpendicular to anatomical axis), may be calculated from the contour. Similarly, ratio “rpapp” may be calculated from the template projection on image plane (Y-Z plane). The angle of rotation is calculated such that the ratio “rpapp” matches with the ratio “rcapp.” After step 1, step 2 is applied to find optimum translation, rotation, and scaling using a shape registration method like ICP, in the same way as it is applied for the anterior-posterior view. If the two images are exactly orthogonal to each other from bi-planar X-ray imaging, refer to FIG. 14.

Instead of separately finding a pose of the template with respect to AP and ML images/contours (as explained above), the template may be aligned in 3D space to match its projection contours, i.e., the template projection contours, with respect to both AP and ML contours simultaneously, using a shape registration method like ICP. Optimal values transformations (translation, scaling, and rotation) may be calculated using ICP, for the template to align it with both the input contours (ML and AP). The camera model with respect to the ML and AP view X-ray image are combined. In the combined camera model, the ML and AP view image planes and image centers have known fixed relative position and known fixed relative orientation (usually 90 degree) with respect to each other. Using this determined relative position and orientation the two camera models (for ML and AP view) are combined in one imaging space and include, two X-ray point sources, two image planes orthogonal to each other, and known SFD (source-film distance). A position of template is found in the imaging space in such a way the template projection contours on both image planes (calculated according to corresponding camera models) aligned with the shape of the corresponding contours. For this, the template is rotated and translated in the imaging space and the optimal rotation and translation parameters are found using modified ICP based method.

Example embodiment system 1 may include a selective anatomical modifier 26 for global matching configured to selectively modify anatomical regions by scaling, translation, and/or rotation to match the 2D projections of its anatomical landmarks, axes, and parameters with the 2D anatomical parameters extracted from the final X-ray image (at least one). This may be done with respect to the ML and AP image for a truncated distal femur or proximal tibia. For example, the corresponding template may be uniformly scaled along all three directions (X, Y, and Z) to match the medial-lateral width of distal femoral condyle or proximal tibial condyle approximately. For a full femur, additional steps may be performed to match the shaft length, shaft axis and neck axis. The template's shaft part region may be scaled along the anatomical axis to match the length of 2D projection of the anatomical axis with the corresponding length in the input X-ray image. The femoral shaft region may be divided into sub-regions along the shaft-axis. The femoral shaft region may be sheared where sub-regions may be translated, bent where sub-regions may be rotated, and/or twisted where sub-regions may be rotated along shaft axis in such a way that the 2D projection of its shaft axis matches with the shaft axis in the input X-ray image. The femoral trochanter, neck, and ball regions (and maybe their sub-regions) may be sheared, scaled, bent, twisted, translated, and rotated along its neck axis to match the positions of the Femoral ball landmark, the Femoral greater trochanter tip landmark in the input X-ray image with the 2D projections of the corresponding landmarks of the template. Similarly, for the full tibia, the shaft length may be matched by scaling the template's shaft part along its anatomical axis to match the length of 2D projection of the anatomical axis with the corresponding length in the input X-ray image. All these operations may be performed while preserving connectivity between parts (neck, ball, shaft etc.).

2D values of the anatomical parameters of extracted from both AP and ML images may then be combined according to the determined camera model to get their 3D values with a 3D geometric calculation mechanism (standard 3D geometry method). The template is then selectively modified where regions or sub-regions may undergo transformations like scaling, shearing, translation, and rotation to match the 3D value of its landmarks, axes and anatomical parameters with the 3D values of the anatomical parameters calculated from the 2D values extracted from the AP and ML images.

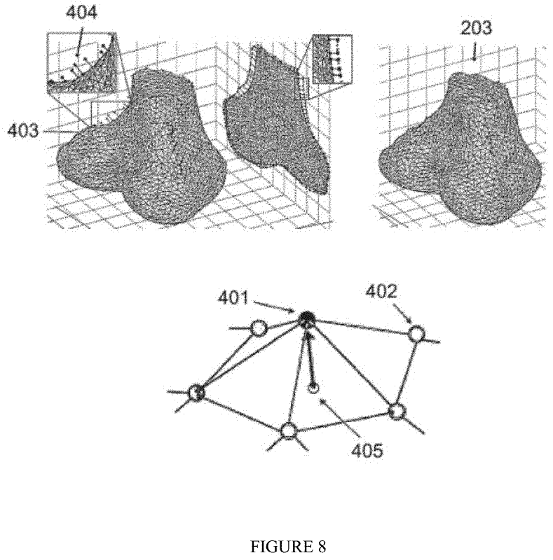

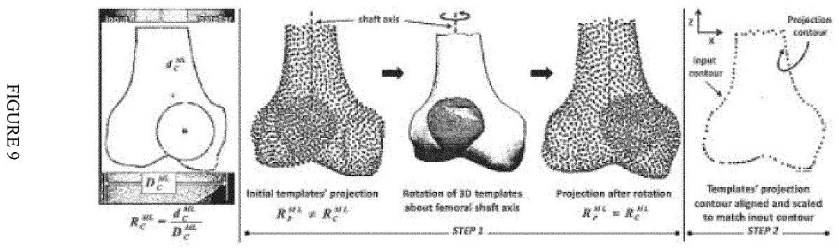

Example embodiment system 1 may include a template deformer 18e configured to deform a standard template model in accordance with defined contours and silhouette vertices obtained from the bi-planar X-ray images. Deformation may include deforming the transformed template mesh in such a way that the silhouette vertices get their target position (which will be determined using a SOM technique explained below) while preserving the overall topology and differential property of the transformed template. FIG. 8 illustrates deformation using Laplacian surface deformation (LSD). Each vertex of a mesh 401 is represented as a differential coordinate, which is the difference between the position of vertex and that of its neighbor vertices 402. In general, the inputs are the initial mesh, a set of anchor points (a few vertices of the initial mesh) and target positions of the anchor points. The output is a deformed mesh where the anchor points take the target positions while preserving the local shape features and topology of the initial mesh. For the template deformation, the template mesh model may be input as the initial mesh, the silhouette vertices with initial positions 403 are the anchor points, and the target positions 404 of the silhouette vertices are the target positions of the anchor points. The differential coordinate 405 for each vertex 401 is defined as the vector from the coordinates of the centroid of its immediate neighbors to its coordinates.

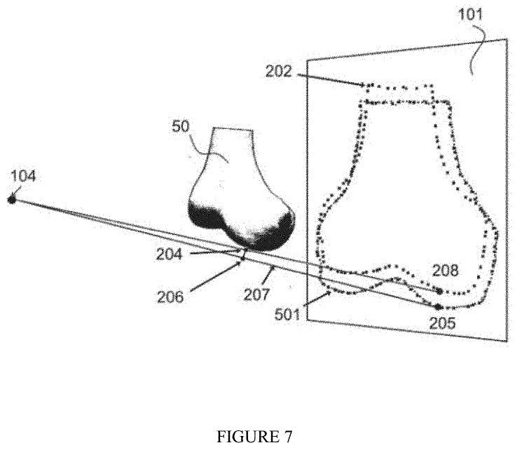

The template deformation may be performed using a Laplacian Surface Deformation (LSD) based method. As seen in FIG. 7, the template projection contour points may be adapted to the input contour using a self-organizing maps (SOM) technique. The top contour is template projection contour 202. Black contour is the input contour. The lower contour is the adapted template projection contour 501 obtained by deforming template projection contour 202 using a SOM technique. This is how to find 2D-2D correspondence. By back projecting the points of these adapted template projection contour, desired positions of the silhouette vertices are obtained and hence the 3D-3D correspondence is obtained. This 3D-3D correspondence may then be used to deform the 3D template using a Laplacian Surface Deformation technique. The SOM technique smoothly deforms the projection contour and preserves the topology (connectivity).

In SOM, for each point of the input contour, the nearest point of the projection contour may be identified and partially pushed toward the contour point. The neighboring points of that particular projection point may also be pushed toward the input contour point. However, their motion is controlled by a specific neighborhood which is an exponential function whose value is high for the projection contour points that are closer to the winner and small for points which are farther away. The adaptation process lessens smoothly with time and controlled by another exponential function called learning rate. SOM gives the 2D-2D correspondence—template projection contour points—adapted template projection contour points between template projection contour 202 and adapted template projection contour 501.

From the 2D-2D correspondence, 3D-3D correspondence point pairs may be calculated for the silhouette vertices by the back projection method of FIG. 5. Using back projection, the adapted template projection points were back projected to find target positions of corresponding silhouette vertices. The silhouette vertices—their target positions—may be the 3D-3D point pairs. The 3D-3D point pairs may be used as positional constraints for LSD. The inputs of the LSD were the template mesh, the silhouette points which will act as the anchor points, and target positions of the silhouette points which were included in the 3D-3D point pairs. Each vertex of the mesh is represented by the differential coordinate that is a difference between the position of a vertex and the centroid of the neighboring vertices in the mesh. In LSD, the anchor points are forced towards their targets while preserving the differential property of the mesh vertices, causing smooth deformation with preservation of shape features.

Further in deformation, a matching point analysis may compute and provide at least a best matching point, for each of the template projection contour point(s) that correspond to the silhouette vertex position(s), on the input contour of the bone, such as 2D-2D correspondence using the SOM method. Deformation may further include constructing a correspondence map for converting points from the 2D projection of the template to a 3D format. The correspondence depends on the back projection mechanism and method.

After the initial alignment of the template model, a 2D-3D correspondence is determined between the defined points of the 2D input contour and the silhouette vertices of the aligned 3D template model for both ML and AP planes, potentially simultaneously. Using this 2D-3D correspondence, the silhouette vertices may be updated to new positions (target positions) such that their projection, i.e., template projection contour, matches with the input contour. First, a 2D-2D correspondence between the points of template projection contour points and the input contour points is found. A non-rigid registration approach of SOM may be used instead of rigid registration-based method like ICP technique because the ICP technique can give wrong correspondence for complex contour shapes.

One of the non-rigid registration methods based on Kohonen self-organizing maps technique was successfully applied by Ferrarini et al. in their GAMES approach to find 3D-3D shape correspondence, which is like example methods and embodiments to find 2D shape correspondence. The template projection contour points (pp) may be adapted onto the input contour points (pc) using the SOM technique. After the adaptation, the template projection contour points represent the shape of the input contour. The number of the template projection contour points and their topology (connectivity) is preserved in the SOM technique. Hence, the positions of the template projection contour points before and after the adaptation gives the required 2D-2D correspondence. The use of the SOM technique allows smooth changes in the shape formed by the template projection contour points.

In an example method, for each input contour point, a best matching template projection contour point ppwinner—a point nearest to the input contour point—may be determined and its position updated toward the input contour point. When the template projection contour adapts to the input contour, the motion of the best matching template projection contour point ppwinner affects a neighbor template projection contour points as well. This is controlled by the neighborhood function n(ppwinner, ppm), which is an exponential function whose value is high for the template projection contour points that are closer to the ppwinner and small for points which are farther away. The neighborhood function is responsible for topology preservation during the adaptation. The adaptation of all the projection contour points is performed with respect to every input contour point. The adaptation of every template projection contour point and its effect on the neighbor points decrease exponentially. This is controlled by the learning rate l(t), which is a function that makes the adaptation process die smoothly with time. In the system and method of this invention, the learning rate constant decreases from 0.5 to 0.1. The whole process, including adaptation of template projection contour points with respect to all the input contour points may also be repeated through number of cycles (iterations) until the MAD value between the points of template projection contour and their corresponding closest points of the input contour goes below a threshold, such as 0.15 mm for example.

The output of SOM technique is the adapted template projection contour points (ppl) onto the input contour. The template projection contour points before and after the adaptation represents the required 2D-2D correspondence. As the template projection contour points are directly associated with the silhouette vertices (projection), the 2D-2D correspondence showing which template projection contour point corresponds to which input contour point directly gives the required 2D-3D correspondence of which silhouette vertex of template corresponds to which input contour point.

Using the 2D-3D correspondence, the silhouette vertices may be updated to their target positions in such a way that their projections represent the shape of the input contours. The corresponding target positions vs1 of the m^{th} silhouette vertices of the template with initial positions vs are determined using the same 3D-3D point pair calculating method (back projection) used for template alignment as shown in FIG. 5. For an^{mth} adapted template projection contour point pmp 1 lying on the input contour, a projection ray rm is determined starting from the X-ray point source meeting the point pmp1 itself. A new position vmsl closest to a corresponding m^{th} silhouette vertex with initial position vsm is found on the updated projection ray. The new position vmsl is the target positions of the m^{th} silhouette vertices. During template deformation, the silhouette vertices may be updated to their target positions, according to which all other vertices of the template are also updated while preserving the overall shape features. This procedure of template deformation is carried out using Laplacian surface deformation. In an example of deformation, a projection and positioning may back-project each of the best matching point(s) to find a position on the back-projected X-ray that is closer to the corresponding silhouette vertices where the target position of each silhouette vertex: 3D-3D correspondence.

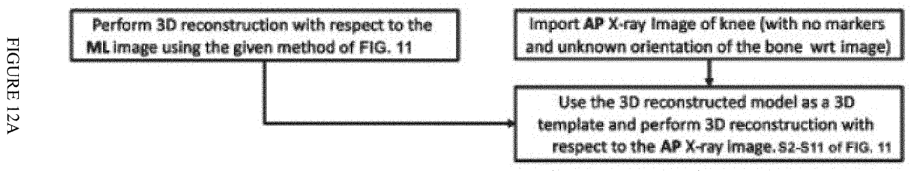

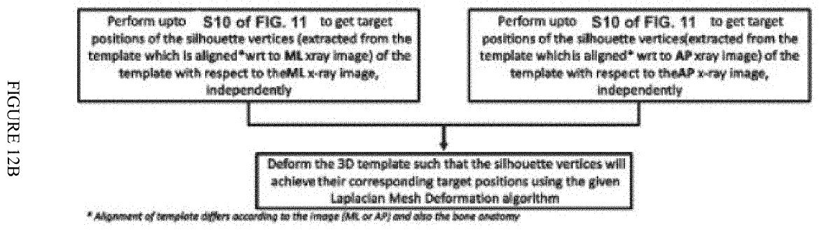

FIG. 11 illustrates a flowchart of 3D image reconstruction from a single X-ray image. As shown in FIG. 11 A first X-ray is taken keeping the bone in its first pre-determined position with the X-ray source to image distance being known. Typically, the first pre-determined position for the first X-ray is such that an anterior-posterior X-ray is taken. A second X-ray is taken keeping the bone in its second pre-determined position with the X-ray source to image distance being known. Typically, the second pre-determined position for the second X-ray is such that a medial-lateral X-ray is taken. Typically, the second X-ray is orthogonally angularly displaced with respect to the first X-ray, about the axis of the bone.

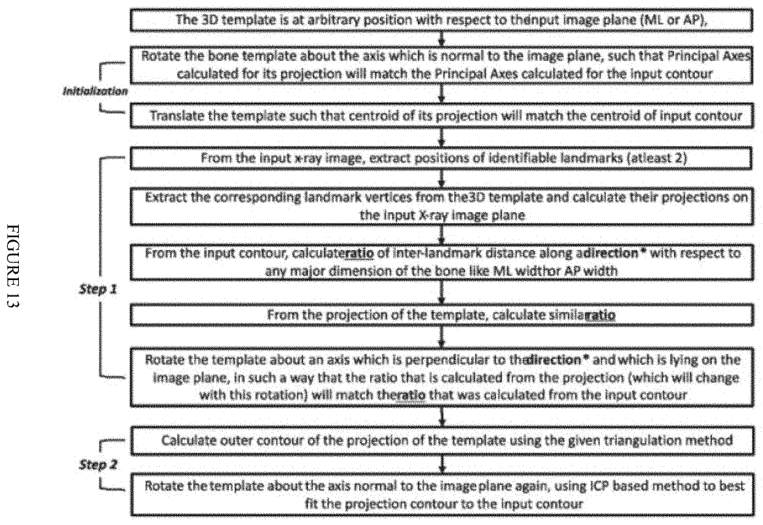

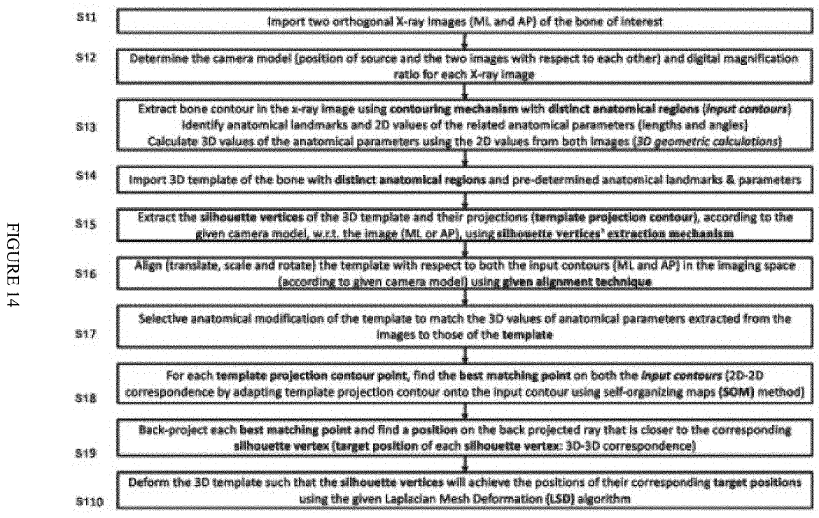

FIG. 12A illustrates an example method of 3D image reconstruction and template deformation separately with respect to ML and then AP X-ray image. FIG. 12B illustrates an example method of the 3D image reconstruction and template deformation simultaneously with respect to ML and then AP X-ray image. FIG. 13 illustrates an example method of determining alignment of the template with respect to the input X-ray image. FIG. 14 illustrates an example method of 3D image reconstruction from a two Orthogonal X-ray image.

Example systems and methods may include view manipulation to manipulate views of the rendered and deformed 3D template. This may enable a user to carry out any or more of: rotate, pan, zoom the view using touch based user input; display or hide individual bones, such as display or hide femur from knee joint, using touch based inputs; cut sectional view of each bone; and/or change color and transparency of individual bone using touch based inputs. A user or a surgeon may virtually plan a surgery using the manipulate views of the rendered and deformed 3-dimensional template. Surgery planning tools may allow the user or the surgeon to plan the surgery, virtually. A surgeon can now use the 3D view of the bone/joint anatomy to plan certain surgeries by manipulating the 3D bone models or importing 3D models of bone implants (depending on the surgery) onto the rendered image. The manipulations May include: rotate/translate the 3D bone model about/along all the 3 axes of the Cartesian coordinate system using touch inputs; resect/Cut the bone into segments and rotate or translate the individual segments using various options provided; select the landmark points (regions) on the 3D bone surface; and/or import 3D models (in STL format) of bone implants onto the 3D interface of the software application.

Example systems and methods may thus enhance portability. Conventional process of planning the surgery use hard copies of X-ray image of the particular region of the patient's body which has to be operated and does not allow a surgeon to simulate the post-operative conditions and it is inconvenient for measurements. Example embodiments and methods use digital X-ray images that can be handled on a portable tablet; a portable method of surgery planning where the surgery plan/simulation can be easily referred during the surgery in the operation theatre. Example systems and methods allow planning of the surgery in 3D view of bone/joint anatomy, which requires only 2-dimensional X-ray images of a patient. Prior art techniques to obtain a 3D model of bones uses CT scans as input and patient has to undergo CT scanning. Thus, example systems and methods require only low cost 2D X-ray images which have about 20 times less cost than a CT scan, the input X-ray images can be acquired by the normal routine procedure of X-ray images with conventional single view imaging equipment; biplanar X-ray imaging equipment or exact orthogonal views of images are not required; 2D X-ray images have around 500 times less radiation than CT scans, lessening patient exposure; 2D X-ray imaging equipment is more prevalent and less expensive than CT scan equipment; and CT scan data is much larger, complicating handling and communication. The ability to use example systems and methods on smaller or tablet devices helps in accurate planning/simulation of the surgery; the tablet interface enables a portable process with a touch-based user interface with easier interactive, touch-based 3D view manipulation of 3D models and views. Case studies can be easily saved in the mobile tablet device and can be shared and archived and 3D models can be printed. Example methods of 2D to 3D conversion based on Laplacian deformation may provide a more efficient shape deformation technique.

Some example methods being described here, it is understood that one or more example methods may be used in combination and/or repetitively to produce multiple options and functionalities for users of communications devices. Example methods may be performed through proper computer programming or hardware configuring of networks and communications devices to receive augmented reality, origin, and limitation information and act in accordance with example methods, at any number of different processor-based devices that are communicatively connected. Similarly, example methods may be embodied on non-transitory computer-readable media that directly instruct computer processors to execute example methods and/or, through installation in memory operable in conjunction with a processor and user interface, configure general-purpose computers having the same into specific communications machines that execute example methods.

Example methods and embodiments thus being described, it will be appreciated by one skilled in the art that example embodiments may be varied through routine experimentation and without further inventive activity. For example, example embodiments have been described in connection with leg bones, it is understood that vastly different anatomy may be used in the same. Variations are not to be regarded as departure from the spirit and scope of the exemplary embodiments, and all such modifications as would be obvious to one skilled in the art are intended to be included within the scope of the following claims.

We claim:

1. A method for obtaining 3-dimensional images from x-ray information for deformed bones, said bones having a distal condyle segment, a proximal condyle segment, and a shaft segment, said method comprising the steps of:
acquiring at least an x-ray image of a bone of interest;
determining camera model, of said acquired at least an x-ray image, using known parameters to at least determine spatial values of source and to determine at least one spatial value of bone;
acquiring contour points and landmark points, in a 2-dimensional co-ordinate system, of said bone, from said image;
transforming said contour points and said landmark points from said 2-dimensional coordinate system into a 3-dimensional co-ordinate system of an imaging space, using said camera model;
importing a pre-created 3-dimensional template corresponding to said bone along with anatomical values, anatomical regions, anatomical landmarks, and anatomical axes;
segmentation of said 3-dimensional template into various anatomical regions;
identification of said anatomical landmarks of said 3-dimensional template;
aligning 3-dimensional template using said identified anatomical landmarks and said segmented anatomical regions with respect to said camera model in order to obtain an aligned template; and
deforming said aligned template, at various regions, to reconstruct deformity in said bone of interest, in such a way that its projection on an X-ray image plane in said imaging space matches exactly with said acquired contour points.

2. The method as claimed in claim 1 wherein, said pre-created 3-dimensional template comprising a 3-dimensional bone model in the form of mesh (object with vertices and faces) with triangular elements, vertices of said mesh representing points on surface of said bone.

3. The method as claimed in claim 1 wherein, said deformed aligned template comprising a 3-dimensional bone model in the form of mesh (object with vertices and faces) with triangular elements, vertices of said mesh representing points on surface of said bone, said mesh comprising new deformed and aligned mesh values.

4. The method as claimed in claim 1 wherein, said step of template segmentation comprising the steps of:
computing three principal axes by applying principal component analysis on vertices 3-dimensional position data of said template;
rotating said template such that first principal axis matches with Z-axis, second principal axis with X axis, and third principal axis with Y axis;
separating middle vertices of said template along Z-axis and finding a best-fit cylindrical axis to said vertices;
realigning said template such that said best-fit cylindrical axis becomes parallel to Z-axis;
extracting bottom vertices of said template along Z-axis as distal condyle segment;
extracting top vertices of said template along Z-axis as proximal condyle segment; and
extracting remaining vertices of said template along Z-axis as shaft segment.

5. The method as claimed in claim 1 wherein, said step of landmark identification comprising a step of computing landmarks based on standard directions of said bone's anatomical coordinate system.

6. The method as claimed in claim 1 wherein, said anatomical parameters defining deformity in said elongate bone, in said anatomical coordinate system, said anatomical parameters being calculated along standard directions, said directions being at least one of Anterior-Posterior (AP), Medial-Lateral (ML), and Superior-Inferior (SI).

7. The method as claimed in claim 1 wherein said step of transforming said contour points and said landmark points from said 2-dimensional co-ordinate system into a 3-dimensional co-ordinate system comprising the steps of using calibration parameters for X-ray images followed by alignment of said template in the 3-dimensional imaging space.

8. The method as claimed in claim 1 wherein, said calibration parameters comprising position of X-ray source, source film distance, principal point position, said calibration parameters being in a 3-dimensional space.

9. The method as claimed in claim 1 wherein, said contour points comprising boundary of selected regions of said bone which represent torsional deformity and joint deformity, said contour points, being in a 2-dimensional co-ordinate system, extracted from a 2-dimensional X-ray image.

10. The method as claimed in claim 1 wherein, said landmark points comprising essential landmarks of said bone to represent its deformity, said landmark points, being in a 2-dimensional co-ordinate system, extracted from a 2-dimensional X-ray image.

11. The method as claimed in claim 1 wherein, said template comprising a 3-dimensional bone model in the form of mesh (object with vertices and faces) with triangular elements, vertices of said mesh representing points on surface of said bone.

12. The method as claimed in claim 1 wherein said deforming being at least one of shaft bending deformity, condyle region deformity, and torsional deformity.

13. The method as claimed in claim 1 wherein, said method comprising a step of computing anatomical axes from said anatomical regions.

14. The method as claimed in claim 1 wherein, said method comprising a step of computing positions of anatomical landmarks based on final X-ray image with respect to extracted contours, based on anatomical regions.

15. The method as claimed in claim 1 wherein, said method comprising a step of computing anatomical parameters based on anatomical landmarks, positions of anatomical landmarks based on final X-ray image with respect to extracted contours, based on anatomical regions, wherein parameters can be a distance between two landmarks, an angle between lines defined by any two landmarks, and/or any correlative value between landmarks.

16. The method as claimed in claim 1 wherein, said step of deforming said template comprising a further step of initial template alignment and condyle deformation, said further step comprising iterations between the following additional steps:
scaling of said template such that width of projection of said distal condyle segment on said image plane along a joint line matches the same calculated from acquired landmark points;
non-rigid deformation of said distal condyle segment in such a way that the angle between anatomical axis and said joint line in its projection matches the angle calculated from acquired landmark points; and
3-dimensional transformation of said template in such a way that the boundary of projection of its condyle segment onto said image plane best-fits with the respective acquired contour points; and
stopping said iteration average point-to-point distance between the boundary of the projection and the respective contour do not change, thereby providing an accurate condyle deformation as well as accurate alignment of said template in 3-dimensional imaging space

17. The method as claimed in claim 1 wherein, said step of deforming said template comprising a further step of template shaft deformation, said further step comprising the following additional steps:
building 3D reference shaft axis from input shaft axis landmarks;
deforming template shaft segment to match anatomical axis; and
twisting of template shaft segment.

18. The method as claimed in claim 1 wherein, said step of deforming said template comprising a further step of template shaft deformation, said further step comprising the following additional steps:
computation of template shaft axis and deforming the shaft segment such the template shaft axis matches with a reference shaft axis;
dividing said template shaft segment is divided into a plurality of sub-segments along its first principal axis, the centroids of vertices belonging to an open boundary of shaft segment mesh are calculated at its distal and proximal ends (boundary-centroids), the template shaft axis is computed as a curve with a first pre-defined number of uniform points passing through centroid of each sub-segment and the boundary-centroids, the shaft segment vertices are re-divided into a second pre-defined number of sub-segments, lesser than said first pre-defined number of uniform points, based on their positions with respect to the second pre-defined number line segments of template shaft axis;
computing affine transformations for each second pre-defined number line segments of the template shaft axis so that they coincide with corresponding second pre-defined number line segments of the reference shaft axis, the same transformations being applied to associated shaft sub-segments; and
the first pre-defined number being sufficiently large to get a smoothly deformed (bending deformity) shaft segment, thereby reconstructing bending and torsional deformity in said bone.

19. The method as claimed in claim 1 wherein, said step of deforming said template comprising a further step of local deformation, said further step comprising the following additional steps:
smoothly deforming said mesh while bringing a few selected anchor points of the mesh to respective target positions (positional constraints) and maintaining the inter-vertices positional relationship described by co-ordinates, the anchor points being silhouette points of the template bone, said silhouette points being projected on image planes and their corresponding contour points being identified based on self organizing maps; and
back-projecting a ray from each corresponding contour point to the source and a nearest position on the ray from each silhouette point, this nearest position eing the target position for the silhouette point (anchor), thereby resulting in an accurate surface reconstruction of the bone.

20. The method as claimed in claim 1 wherein, said step of obtaining calibration parameters comprises a step of obtaining said calibration parameters from a calibrator.

21. The method as claimed in claim 1 wherein, said step of obtaining contour points, comprises a step of obtaining said contour points from a contourer, from 2D X-ray images.

22. The method as claimed in claim 1 wherein, said step of obtaining landmark points, comprises a step of obtaining said landmark points using camera model determinator, from 2D X-ray images.

23. The method as claimed in claim 1 wherein, said step of obtaining a 3-dimensional bone template, comprises a step of obtaining said 3-dimensional bone from a bone template model inputter.
